(19) United States
(12) Patent Application Publication
(10) Pub. No.: US 2015/0223988 A1
(43) Pub. Date: Aug. 13, 2015

(54) BANDAGE WITH A COMPRESSED LAYER THAT EXPANDS UPON CONTACT WITH LIQUID

(71) Applicant: Donald SPECTOR, New York, NY (US)

(72) Inventor: Donald SPECTOR, New York, NY (US)

(21) Appl. No.: 14/693,049

(22) Filed: Apr. 22, 2015

Related U.S. Application Data
(63) Continuation-in-part of application No. 13/795,055, filed on Mar. 12, 2013.
Application No. 13/795,055 is a continuation-in-part of application No. 12/752,568, filed on Apr. 1, 2010, now Pat. No. 8,419,668.
(60) Provisional application No. 62/128,474, filed on Mar. 4, 2015.
(60) Provisional application No. 61/310,332, filed on Mar. 4, 2010.

(51) Int. Cl. A61F 13/00; A61F 13/02

(52) U.S. Cl. CPC A61F 13/00063; A61F 13/0206

(57) ABSTRACT

A bandage is formed of a film layer, an adhesive applied to the film layer, and an absorbent layer connected to the film layer. The absorbent layer comprises a compressed fabric that upon saturation with liquid expands to a volume that is at least two or three times the volume of the compressed fabric. The film layer extends in at least two directions beyond edges of the absorbent layer, so that the film layer with adhesive can attach to skin around a wound with the absorbent layer covering the wound. The film layer has folded sections adjacent the absorbent layer, so that the folded sections unfold upon expansion of the compressed fabric. The expansion of the fabric layer upon contact with bodily fluids from a wound pulls the fluids and any infectious agents out of an away from the wound, thus speeding the healing process.

### CROSS-REFERENCE TO RELATED APPLICATIONS

This application claims priority under 35 USC 119(e) of U.S. Provisional Application Ser. No. 62/128,474, filed on Mar. 4, 2015. This application is also a continuation-in-part of U.S. patent application Ser. No. 13/795,055, filed on Mar. 12, 2013, which is a continuation-in-part of U.S. patent application Ser. No. 12/752,568, filed on Apr. 1, 2010 (now U.S. Pat. No. 8,419,668 issued Apr. 16, 2013), which claims priority under 35 USC 119(e) of U.S. Provisional Application Ser. No. 61/310,332, filed on Mar. 4, 2010. The disclosures of all of these prior applications is herein incorporated by reference.

### BACKGROUND OF THE INVENTION

1. Field of the Invention

The present invention relates to a bandage having a layer of compressed absorbent material. In particular, the invention relates to a bandage having a layer of compressed material and which is treated with a antimicrobial substance to kill bacteria in a wound.

2. The Prior Art

Present bandage strips typically consist of a layer of gauze padding connected to a longer strip of adhesive film or fabric. The gauze padding typically has a non-stick layer on its side facing the wound to keep the gauze from sticking to the wound. In addition, some bandages are treated with antimicrobial agents to prevent infection of the wound. For example, U.S. Pat. No. 6,967,261 to Soerens et al. discloses a bandage having a multilayer system connected to an adhesive strip. An antimicrobial agent is supplied to the bandage such that the agent can contact the wound.

While this type of bandage may be useful, some wounds are deep and/or large enough that the antimicrobial agent is insufficient for treating the entire wound. The bodily fluids collect in the wound and can cause infection.

### SUMMARY OF THE INVENTION

It is therefore an object of the invention to provide a bandage having an antimicrobial agent and which also draws fluid away from the wound to speed healing and allow the agent to reach all areas of the wound.

This object is accomplished by a bandage comprising a film layer having a top surface and a bottom surface, an adhesive applied to the bottom surface, and an absorbent layer connected to the bottom surface. The absorbent layer comprises a compressed material that upon saturation with liquid expands to a volume that is at least two or three times the volume of the compressed fabric. The film layer extends in at least two directions beyond edges of the absorbent layer, so that the film layer with adhesive can attach to skin around a wound with the absorbent layer covering the wound. The film layer has folded sections adjacent the absorbent layer, so that the folded sections unfold upon expansion of the compressed material. This way, the expanded material does not put any pressure on the film layer or the wearer's skin, as the film layer expands to accommodate the increased volume of the expanded absorbent layer. The expansion of the absorbent layer upon contact with bodily fluids from a wound pulls the fluids and any infectious agents out of and away from the wound, thus speeding the healing process.

In one embodiment, an antimicrobial agent is applied to the absorbent layer. This can be applied as a film over the absorbent layer, or the antimicrobial agent can be embedded within the fabric of the absorbent layer. The antimicrobial agent can be microencapsulated so that the agent is only released upon contact with liquid. Any suitable antimicrobial agent can be used in the bandage according to the invention.

In a preferred embodiment, the absorbent layer is a compressed woven material. In particular, gauze that has been mechanically compressed to ⅓ its volume is an ideal material for the absorbent layer. Upon contact with fluids from a wound, the gauze absorbs the fluid, expands to 3 times its volume, and pulls the fluid and any infectious microorganisms away from the wound. If antimicrobial agents are present in the fabric, the agents then can neutralize the infection before it becomes symptomatic. To form the absorbent layer, the gauze or other material is compressed only in the vertical direction, so that the expansion occurs only in the vertical direction, i.e., up and away from the wound.

In order to prevent the absorbent layer from sticking to a wound, there can be a non-stick layer connected to a surface of the absorbent layer. The non-stick layer can be formed of any suitable material such as silicone or other polymer. The non-stick layer is formed to be permeable so that the fluids from the wound can pass through to the absorbent layer. The non-stick layer can be formed a screen or a perforated sheet.

In order to enable expansion of the absorbent layer without any pressure from the film layer on the wound, the folded sections of the film layer are not covered with adhesive, or the portions touching the adhesive are treated with an easily releasable material, or the top surface of the film layer is an easily releasable material. This way, the absorbent layer can expand freely. The folded sections can be disposed adjacent to the absorbent layer or can be disposed on top of the absorbent layer. Any suitable configuration of the film and absorbent layer can be used so long as the film expands along with the absorbent layer to allow unfettered expansion until the absorbent layer is fully expanded.

### BRIEF DESCRIPTION OF THE DRAWINGS

Other objects and features of the present invention will become apparent from the following detailed description considered in connection with the accompanying drawings. It is to be understood, however, that the drawings are designed as an illustration only and not as a definition of the limits of the invention.

In the drawings, wherein similar reference characters denote similar elements throughout the several views:

FIG. 1 shows a cross-sectional view of the bandage according to the invention prior to use;

FIG. 2 shows a cross-sectional view of the bandage after application to a wound and expansion of the absorbent layer; and

FIG. 3 shows a cross-sectional view of an alternative embodiment of the bandage according to the invention.

### DETAILED DESCRIPTION OF THE PREFERRED EMBODIMENT

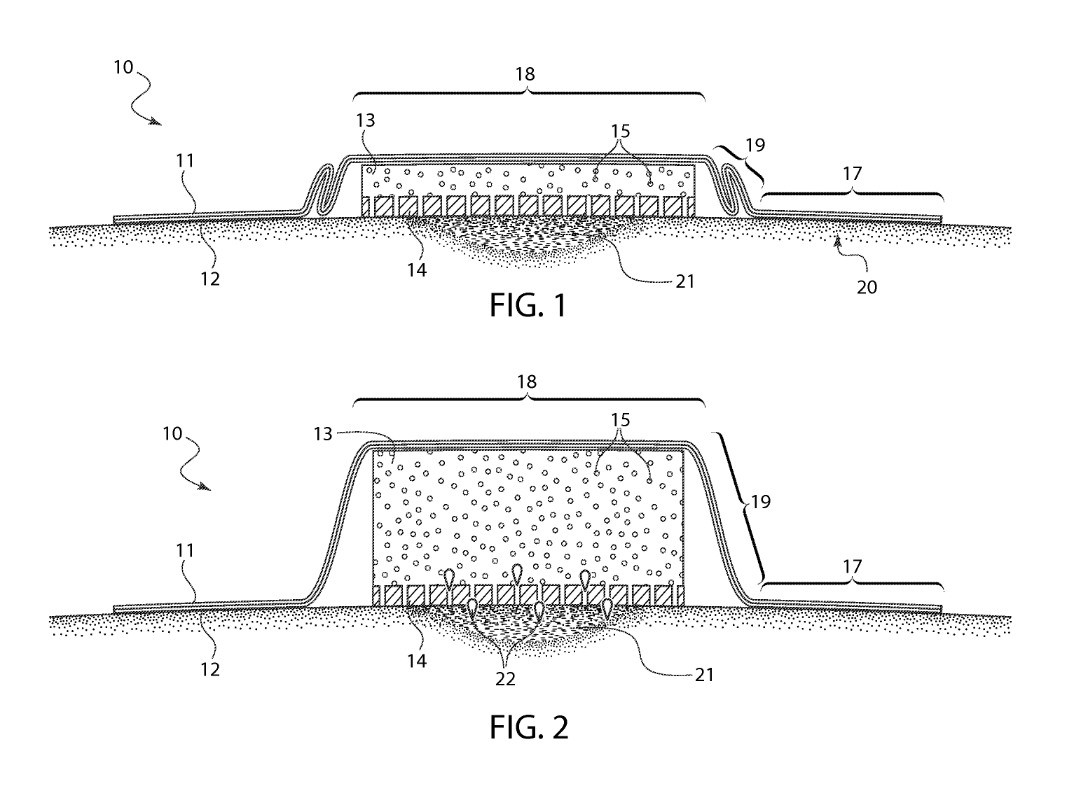

Referring now in detail to the drawings, FIG. 1 shows a cross-sectional view of the bandage 10 according to the invention after application to a patient's skin 20 for covering a wound 21. Bandage 10 comprises a film layer 11, an adhesive layer 12 extending the length of film layer 11, and an absorbent layer 13 disposed centrally along film layer 11, so that end sections 17 of film layer 11 extend beyond absorbent layer 13. A non-stick layer 14 is applied to the bottom of absorbent layer 13 to prevent absorbent layer 13 from sticking to the wound 21. Non-stick layer 14 is water permeable and can be configured as a screen or with perforations to allow fluid to pass through to absorbent layer 13. Non-stick layer 14 can be configured of any suitable material, such as silicone or polypropylene. Film layer 11 can be manufactured from any suitable film material that is commonly used in disposable bandages. Common film materials are extruded polymers, but woven materials could also be used instead of a film.

Embedded within absorbent layer 13 is a microencapsulated antimicrobial agent 15. The antimicrobial agent 15 is configured to be released from microencapsulation upon contact with fluid, so that the agent can mix with the fluid and kill any microorganisms in the fluid.

Absorbent layer 13 is comprised of a compressed material such as foam or woven fabric. The compression is performed via a compression machine to a degree such that upon contact with liquid, the absorbent layer 13 expands to a height that is at least three times the height of the compressed absorbent layer 13. This expansion can be seen in FIG. 2. Upon contact with fluids 22 from wound 21, layer 13 expands in height to at least 3 times the height of the compressed absorbent layer shown in FIG. 1. The preferred degree of expansion is between 3 and 10 times the height of the compressed absorbent layer, but greater or lesser expansion degrees could also work with the present invention. The compression takes place only in the vertical direction, so that upon contact with liquid, the absorbent layer expands only in the vertical direction.

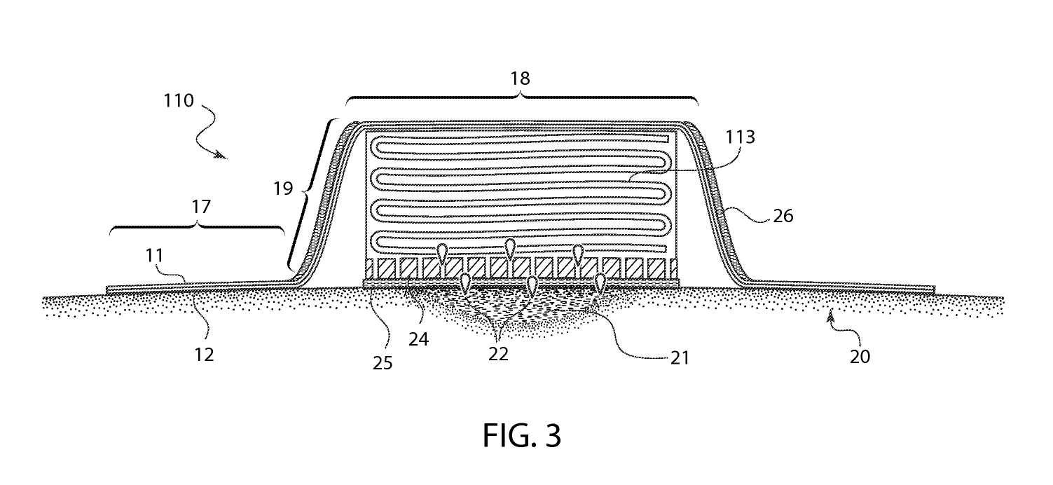

To allow this expansion, the area 19 of film layer 11 that is disposed between end sections 17 and middle section 18 is folded around itself when the bandage 10 is in the compressed state. Once expansion begins, the folded area 19 unfolds to accommodate the increase in height of absorbent layer 13. To allow for the unfolding to occur, the adhesive layer 12 can be removed from section 19, or adhesive layer 12 or the upper part of film layer 11 in this area can be treated with a non-stick substance 26 (shown in FIG. 3) so that the adhesive layer 12 does not prevent unfolding of film layer 11 in section 19 as absorbent layer 13 expands. Or, the entire top surface of film layer 11 can be manufactured non-stick.

During expansion of absorbent layer 13, the fluid 22 is pulled out of wound 21 into absorbent layer 13, where it contacts antimicrobial agent 15 to prevent and destroy any infection in the fluid. Antimicrobial agent 15 can be any suitable agent, such as a topical antibiotic (erythromycin, sulfacetamide sodium, bacitracin, neomycin) or antiseptic (sodium hypochlorite, ethanol, iodine, chlorhexidine). By pulling the fluid out of the wound, bandage 10 prevents infection in the wound and speeds healing.

FIG. 3 shows an alternative embodiment of the bandage according to the invention. Here, bandage 110 comprises film layer 11, adhesive layer 12, absorbent layer 113 and release layer 14, which all function in the same manner as elements 10, 11, 12, 13 and 14 described above with respect to FIGS. 1 and 2. In this embodiment, absorbent layer 113 consists of a woven gauze. Layer 113 is compressed mechanically and then expands to a final height as shown in FIG. 3 upon contact with fluid 22 of wound 21. Instead of microencapsulated antimicrobial agent 15, the embodiment of FIG. 3 has a layer 25 of an antimicrobial agent disposed on the bottom of bandage 110 underneath absorbent layer 113. Upon contact with the wound 21, antimicrobial agent 25 can migrate into the wound 21 as well as mix with fluid 22 and be absorbed up into absorbent layer 113.

Accordingly, while only a few embodiments of the present invention have been shown and described, it is obvious that many changes and modifications may be made thereunto without departing from the spirit and scope of the invention.

What is claimed is:

1. A bandage comprising:
a film layer having a top surface and a bottom surface;
an adhesive applied to the bottom surface; and
an absorbent layer connected to the bottom surface, the absorbent layer comprising a compressed material that upon saturation with liquid expands to a volume that is at least two times the volume of the compressed material,
wherein the film layer extends in at least two directions beyond edges of the absorbent layer, wherein the film layer has folded sections adjacent the absorbent layer, and wherein the folded sections unfold upon expansion of the compressed material.

2. The bandage according to claim 1, further comprising an antimicrobial agent applied to the absorbent layer.

3. The bandage according to claim 1, wherein the compressed material is a woven fabric.

4. The bandage according to claim 2, wherein the antimicrobial agent is in the form of a film applied to a surface of the absorbent layer.

5. The bandage according to claim 2, wherein the antimicrobial agent is microencapsulated and embedded within the compressed material, and wherein the antimicrobial agent is released from encapsulation upon exposure to liquid.

6. The bandage according to claim 1, further comprising a non-stick layer connected to a surface of the compressed layer.

7. The bandage according to claim 1, wherein the folded sections of the film layer are treated with a non-stick layer on a surface opposite the adhesive layer.

8. The bandage according to claim 1, wherein absorbent layer expands to a volume that is at least three times the volume of the compressed material upon saturation with liquid.
